(19) United States
(12) Patent Application Publication
(10) Pub. No.: US 2026/0034039 A1
(43) Pub. Date: Feb. 5, 2026

(54) CLEANSERS

(71) Applicant: Dr. Squatch, LLC, Marina Del Rey, CA (US)

(72) Inventors: Alexandra Blackstone, Redondo Beach, CA (US); Duane Kleingarn, West Hollwood, CA (US); Alexander Uruchurtu, Los Angeles, CA (US); Darlene Rodriguez, Artesia, CA (US)

(21) Appl. No.: 19/288,886

(22) Filed: Aug. 1, 2025

Related U.S. Application Data
(60) Provisional application No. 63/679,070, filed on Aug. 2, 2024.

(51) Int. Cl. A61K 8/36; A61Q 19/00; A61Q 19/10

(52) U.S. Cl. CPC A61K 8/361; A61Q 19/007; A61Q 19/10; A61K 2800/30; A61K 2800/5424

(57) ABSTRACT

Disclosed are cleanser formulations in a gel form including a fatty acid salt as the primary cleansing agent. The cleanser formulations have a pH ranging from 6-11. The cleanser gels can contain at least 90 weight percent of natural or naturally derived ingredients.

This application claims priority to and the benefit of U.S. Provisional Patent Application No. 63/679,070, filed Aug. 2, 2024, the disclosure of which is incorporated by reference in its entirety.

### BACKGROUND

Cleansers such as body washes and hand soaps can be used to clean skin. Some cleanser formulations may require non-naturally derived or non-natural detergents to achieve adequate cleansing without causing skin irritation. However, non-natural or non-naturally derived detergents may not be desirable for some users. Rather, some users may prefer natural body wash and hand soap formulations that include natural soaps such as a fatty acid salt. Such natural soaps can present challenges to formulating a cleanser that, e.g., does not irritate the skin. Accordingly, there is a need for improved formulations that are natural or naturally derived and contain fatty acid salts without non-naturally derived non-soap detergents.

### DETAILED DESCRIPTION

Unless otherwise indicated, all parts and percentages are by weight. Unless otherwise stated or made clear by context, weight percentages in a composition or formulation are provided based on the total amount of the composition or formulation in which they are described.

As used herein, the singular forms “a,” “an,” and “the” include plural referents unless the context clearly dictates otherwise. The word “or” is the inclusive or and is used synonymously with “and/or” such that “A or B” includes embodiments having only A, embodiments having only B, and embodiments having both A and B.

As used herein, a natural ingredient is an ingredient obtained only from a natural source such as a plant, animal, micro-organism or mineral; however, ingredients obtained from fossil fuels are outside the definition of a natural ingredient. Natural ingredients include ingredients obtained from natural sources through physical processes, fermentation reactions occurring in nature and leading to molecules which occur in nature, and other procedures of preparation that do not involve intentional chemical modification.

A naturally derived ingredient is an ingredient of greater than 50% natural origin, by molecular weight, by renewable carbon content, or by other methods used in the art, obtained through chemical and/or biological processes resulting in chemical modification. Chemical modifications falling within the scope of naturally derived ingredient include, for example, saponification, neutralization, hydration, etherification, amination, esterification, amidation, and hydrolysis. The relevant chemical structure of the natural ingredient remaining intact means, by way of example, that the fatty chain acyl structure of a fatty acid remains after esterification or amidation. An example of additional definitions of natural ingredients is provided in ISO 16128-1:2016(E), (International Organization for Standardization. (2016) Guidelines on technical definitions and criteria for natural and organic cosmetic ingredients and products—Part 1: Definitions for ingredients (ISO Standard No. 16128-1:2016(E)); www.iso.org/standard/62503.html) incorporated herein by reference in its entirety.

The natural content of a formulation (also referred to herein as the naturally derived ingredient content) can be calculated based on the extent to which each ingredient, taken separately, meets the definition of a natural ingredient or naturally derived ingredient of greater than 50% natural origin. This may be expressed as a percentage of natural origin as defined above. For example, for a fatty acid amide derived from a fatty acid and “non-natural” amine, the natural content is the percentage of the molecular weight attributable to the fatty acid acyl group. The natural content of the formulation is the mass percentage of all natural ingredients and naturally derived ingredients in that formulation. It is calculated as the sum of the relative concentrations of each ingredient in the formulation multiplied by each ingredient's respective percentage of natural origin. An exemplary method of calculating percentage of natural ingredient content within a formulation is provided in ISO 16128-2: 2017 (E), (International Organization for Standardization. (2017) Cosmetics-Guidelines on technical definitions and criteria for natural and organic cosmetic ingredients-Part 2: Criteria for ingredients and products (ISO Standard No. 16128-2: 2017 (E)); www.iso.org/standard/65197.html), incorporated herein by reference in its entirety.

As used herein, the term “non-natural or non-naturally derived detergents” refers to detergents or compositions that are not naturally derived as defined above. Non limiting examples of non-natural or non-naturally derived detergents include petroleum products and polyalkylene glycol-based, detergents and cleansers, e.g., polyethylene glycol-based and polypropylene glycol-based surfactants, polysorbates, and other non-naturally derived cationic surfactants, zwitterionic surfactants, anionic surfactants, and non-ionic surfactants.

As used herein, “natural soaps” are soaps made by saponification of natural fats and oils, including butters. Suitable natural fats and oils include esters of glycerol having three fatty acid esters, i.e., triglycerides. As such, natural soaps are fatty acid salts, i.e. an alkali metal salt (e.g., a lithium salt, a sodium salt, or a potassium salt), an alkaline earth metal salt (e.g., a magnesium salt or a calcium salt), or salts with other positive ions. Preferably, natural soaps are water soluble or dispersible.

As used herein, a “cleanser” is a substance that may be used to clean the human body and includes, for example, body washes, hand soaps, and face washes.

The use of natural soaps in cleansers, particularly in the absence of other non-natural or non-naturally derived detergents or cleansers, can present challenges to formulating a cleanser in the form of a gel. For example, cleanser formulations that contain a natural soap, e.g., a fatty acid salt, may have a basic pH. A basic pH may present challenges for formulating a cleanser because additives (e.g., rheology modifiers) may not function properly at a basic pH. For example, it may be difficult to formulate a cleanser as a gel when using a natural soap because the basic pH may render the performance of some conventional additives unsatisfactory. For example, common thickeners, such as carbomers or acrylate/acrylic acid based thickeners typically have a low pH to neutral pH range and the efficacy of such components as thickeners may be reduced at other pHs. Some gums and natural thickeners can also be susceptible to degradation at extreme pH levels, e.g., at a pH above 10 or below 3.

Natural soaps may also cause more skin irritation than some non-natural or non-naturally derived detergents. While this may not be problematic for some applications, this tendency to irritate the skin can be problematic for applications where large portions of the skin are exposed to the natural soap. It may therefore be difficult to formulate a cleanser using natural soaps such that the cleanser does not irritate the skin.

Disclosed are cleanser formulations that contain a fatty acid salt as the primary cleansing agent and a moisturizer wherein the cleanser is a gel with a pH suitable to maintain the natural soap in a salt form. “Fatty acid salt as the primary cleansing agent” means that that at least 95% of the cleansing agent components (i.e. surfactants, detergents and cleansers added for cleaning qualities) is a fatty acid salt. In some embodiments, the fatty acid salt is a potassium salt, e.g., potassium cocoate. In some embodiments, the primary cleansing agent is at least 99% fatty acid salt. In some embodiments, the fatty acid salt is the only added cleansing agent. Also disclosed are body wash formulations that contain a cleansing agent that includes a fatty acid salt, have a dynamic viscosity at 25° C. and 1 atmosphere of pressure that ranges from 4,000 centipoise to 36,000 centipoise as determined with a Brookfield RV viscometer having a RV-5 spindle at 10 rpm, have a pH between 6 and 11, and have at least 90 weight percent of natural or naturally derived ingredients.

Further disclosed are hand soap formulations that contain a cleansing agent that includes a fatty acid salt, have a dynamic viscosity at 25° C. and 1 atmosphere of pressure that ranges from 1,000 centipoise to 10,000 centipoise as determined with a Brookfield RV viscometer having a RV-4 spindle at 10 rpm, have a pH between 6 and 11, and have at least 90 weight percent of natural or naturally derived ingredients. In some of these embodiments, the fatty acid salt is the only added cleansing agent.

In some embodiments, the cleanser may also contain naturally derived detergents and surfactants. Naturally derived detergents and surfactants can include sulfates (e.g., sodium lauryl sulfate, sodium glucan sulfate), betaines, (e.g., cocamidopropyl betaine, lauryl betaine and coco-betaine), organic sulphonates (e.g., sodium C14-16 alpha olefin sulfonate), fatty acid and fatty alcohol glucosides (e.g., coco glucosides), alkanolamides (e.g., di and mono-alkanolamides such as coconut diethanolamide and coconut monoethanolamide), isethionates, taurates, and hydroxysultaines, so long as those ingredients meet the definition of a naturally derived ingredient above.

Generally, the formulations will have a pH of 6-11. In some embodiments, the pH ranges from 6.5-10. In some embodiments, the pH of the cleanser ranges from 8-11 or 8-10. In some embodiments, the pH of the cleanser is about 6.5. In the absence of non-natural or non-naturally derived detergents, natural soaps can also be difficult to fully rinse off of the skin. This can result in the perception of having a film remaining on the skin after use. The present formulation overcomes these and other difficulties that have hindered the ability to provide a cleanser gel with a suitable viscosity, feel, compatibility, and rinsability, and without having skin sensitivity or irritability.

### Gels, Viscosity Modifiers, and Liquid Embodiments

The disclosed cleansers can be formulated as a gel. A gel can be characterized by its viscosity. In some embodiments, the cleanser is a body wash gel having a dynamic viscosity at 25° C. and 1 atmosphere of pressure that ranges from 4,000 centipoise to 36,000 centipoise as determined with a Brookfield RV viscometer having a RV-5 spindle at 10 rpm. In some embodiments, the body wash is a gel having a dynamic viscosity that ranges from 8,000 centipoise to 32,000 centipoise as measured above. In some embodiments, the body wash is a gel having a dynamic viscosity that ranges from 10,000 centipoise to 32,000 centipoise as determined by the above method. In some embodiments, the body wash is a gel having a dynamic viscosity that ranges from 12,000 centipoise to 32,000 centipoise as determined by the above method. In some embodiments, the body wash is a gel having a dynamic viscosity that ranges from that ranges from 14,000 centipoise to 32,000 centipoise as determined by the above method. In some embodiments, the body wash is a gel having a dynamic viscosity that ranges from 16,000 centipoise to 32,000 centipoise as determined by the above method. In some embodiments, the body wash is a gel having a dynamic viscosity that ranges from 18,000 centipoise to 32,000 centipoise as determined by the above method. In some embodiments, the body wash is a gel having a dynamic viscosity that ranges from 20,000 centipoise to 32,000 centipoise as determined by the above method. In still further embodiments, the body wash is a gel having a dynamic viscosity that ranges from 10,000 centipoise to 12,000 centipoise as measured above.

In further embodiments the cleanser can be a hand soap have a dynamic viscosity at 25° C. and 1 atmosphere of pressure that ranges from 1,000 centipoise to 10,000 centipoise as determined with a Brookfield RV viscometer having a RV-4 spindle at 10 rpm. In some embodiments, the hand is a gel having a dynamic viscosity that ranges from 7,000 centipoise to 8,000 centipoise as measured above.

It should be noted that the viscosity ranges given above are determined with the indicated Brookfield RV viscometer equipped with a RV-4 or RV-5 spindles rotating at the given speeds. As will be appreciated by those skilled in the art, measuring viscosity using different equipment and/or different procedures may result in a differently determined viscosity values.

The cleanser can be formulated as a gel by using a viscosity modifier. For example, in some embodiments, the body wash contains a viscosity modifier chosen from water soluble polysaccharides (in particular, gums), complex starches, cellulosic thickeners, acrylate-based polymers, and combinations thereof. Suitable viscosity modifiers are stable and provide efficacious thickening properties at a pH in the range of 6-11, and particularly in the range of 8-11.

In still other embodiments, the cleanser is formulated as a liquid soap that does not include a thickening agent so as to be water thin. Such a liquid soap could be, for example, a foaming hand soap that is dispensed with a pump mechanism.

As discussed further below, some embodiments of the invention include suspended exfoliants. Exfoliants in a liquid formulation do not remain suspended, but can settle out and sink to the bottom. Accordingly, particularly when exfoliants are present, the viscosity modifier must have a sufficient yield value to maintain the exfoliant particle in suspension. Yield value is commonly defined as initial resistance to flow under applied stress. In particular, the minimum shear stress required to initiate flow is known as the yield value or yield index. When the force of gravity operating on a suspended particle exceeds the liquid's yield value, the particle will not descend. If the yield value is insufficient, particles suspended in a liquid will tend to settle.

While some thickening agents and viscosity modifiers can provide thickening efficiency, they do not have a sufficient yield value to maintain particles in suspension. For example, inorganic sodium and potassium salts, e.g., sodium chloride and or potassium chloride, have been added to thicken solutions of fatty acid salts. This can be effective when the fatty acid salt is a sodium fatty acid salt or a mixture of sodium and potassium fatty acid salts. In some embodiments, the cleanser does not contain any added inorganic sodium or potassium salts. Glycerides and triglycerides have also been used as gelling agents in soap formulations. In some embodiments, the cleanser gel is free of added glycerides and triglycerides other than those that may be present as part of a fragrance. Exemplary viscosity modifiers having satisfactory thickening properties and sufficient yield value include water soluble polysaccharides such as gums, e.g., xanthan gum.

In some embodiments, the cleanser includes from 0.01 weight percent to 2 weight percent of the viscosity modifier by total weight of the cleanser. For example, in some embodiments, the cleanser includes from 0.01 weight percent to 2 weight percent of xanthan gum by total weight of the cleanser.

In some embodiments, the cleanser contains a natural gum as a viscosity modifier. Any natural gum that is stable in the pH range of the cleanser is acceptable. In some embodiments, the cleanser contains xanthan gum as a viscosity modifier. Xanthan gum provides viscosity modification and maintains a sufficient yield and does not negatively impact clarity of the cleanser. In some embodiments, the cleanser contains xanthan gum along with secondary viscosity modifiers. The secondary viscosity modifier may be an additional natural gum, a complex starch, or a cellulosic thickener. In some embodiments, the cleanser contains xanthan gum as the only viscosity modifier. In some embodiments, the cleanser includes less than 1.5% of complex starch, cellulosic thickeners, acrylate-based polymers, or combinations thereof as a viscosity modifier. In some embodiments, the cleanser includes less than 1% of complex starch, cellulosic thickeners, acrylate-based polymers, or combinations thereof as a viscosity modifier. In some embodiments, the cleanser does not include any complex starch, cellulosic thickener or acrylate-based polymer as a viscosity modifier.

### Fatty Acid Salts

The cleanser contains a fatty acid salt, or combination of fatty acid salts, as the primary cleansing agent. Some fatty acid salts are examples of natural or naturally derived soaps. Fatty acid salts include alkali metal salts (e.g., a lithium salt, a sodium salt, or a potassium salt), alkaline earth salts (e.g., a magnesium salt or a calcium salt), or fatty acid salts with other cations. Calcium and magnesium fatty acid salts are generally less water soluble and can cause difficulty in rinsing from the skin, resulting in the perception of having a film remaining on the skin after use. Accordingly, in embodiments, the fatty acid salt is an alkali metal salt. In some embodiments the fatty acid salt is a potassium salt. The fatty acid portion of the fatty acid salt may be any fatty acid (e.g., any naturally occurring fatty acid). For example, in some embodiments, the fatty acid salt has from 4 to 28 carbon atoms and/or the fatty acid salt has an even number of carbon atoms. In some embodiments, the fatty acid salt has from 8 to 22 carbon atoms and/or the fatty acid salt has an even number of carbon atoms. In some embodiments, the fatty acid salt is unbranched. In some embodiments, the fatty acid salt is saturated. In some embodiments, the fatty acid salt is unsaturated. In some embodiments, the fatty acid salt is derived from a natural oil, e.g., coconut oil, cocoa butter or palm oil. In some embodiments, the fatty acid salt is a cocoate salt, an oleate salt, a palmate salt, and combinations thereof. For example, in some embodiments, the fatty acid salt is chosen from potassium cocoate, potassium oleate, potassium palmate, and combinations thereof.

The fatty acid salt may be present in the cleanser at different amounts and may be added as an aqueous solution. The aqueous solution may contain from about 25 weight percent to about 45 weight percent of the fatty acid salt or a mixture of fatty acid salts. In embodiments where the fatty acid is added as an aqueous solution, the aqueous solution is added in an amount of from about 5 weight percent to about 40 weight percent by total weight of the cleanser. For example, in some embodiments, the aqueous solution is added in an amount of from 15 weight percent to 40 weight percent by total weight of the cleanser. In some embodiments, the aqueous solution is added in an amount of from 25 weight percent to 40 weight percent by total weight of the cleanser. In some embodiments, the aqueous solution is added in an amount of from 35 weight percent to 40 weight percent, for example about 37 weight percent by total weight of the cleanser.

A natural soap can comprise a fatty acid potassium salt, can be present in the cleanser at different amounts and can be added as an aqueous solution. In some embodiments, the fatty acid salt contains only species of potassium fatty acid salt, e.g., potassium cocoate. The aqueous solution may contain from about 25 weight percent to about 45 weight percent of the fatty acid potassium salt or a mixture of fatty acid salts. In some embodiments, the aqueous solution of potassium fatty acid salt is added in an amount of from about 5 weight percent to about 40 weight percent by total weight of the cleanser. For example, in some embodiments, the aqueous solution of potassium fatty acid salt is added in an amount of from 15 weight percent to 40 weight percent by total weight of the cleanser. In some embodiments, the aqueous solution of potassium fatty acid salt is added in an amount of from 25 weight percent to 40 weight percent by total weight of the cleanser. In some embodiments, the aqueous solution of potassium fatty acid salt is added in an amount of from 35 weight percent to 40 weight percent, for example about 37 weight percent by total weight of the cleanser.

In some embodiments, the natural soap may comprise potassium cocoate. The potassium cocoate may be present in the cleanser at different amounts and may be added as an aqueous solution. The aqueous solution may contain from about 5 weight percent to about 50 weight percent of potassium cocoate. For example, in embodiments where the cleanser is a body wash, the aqueous solution may contain from about 25 weight percent to about 50 weight percent of potassium cocoate. In other examples where the cleanser is a hand soap, the aqueous solution may contain about 5 weight percent potassium cocoate. In such embodiments, the aqueous solution of potassium cocoate is added in an amount of from about 5 weight percent to about 40 weight percent by total weight of the cleanser. For example, in some embodiments, the potassium cocoate is added in an amount of from 15 weight percent to 40 weight percent by total weight of the cleanser. In some embodiments, the aqueous solution of potassium cocoate is added in an amount of from 25 weight percent to 40 weight percent by total weight of the cleanser. In some embodiments, the aqueous solution of potassium cocoate is added in an amount of from 35 weight percent to 40 weight percent, for example about 37 weight percent by total weight of the cleanser.

In some embodiments, the aqueous solution is a solution containing about 25 weight percent to about 40 weight percent potassium cocoate and about 2 weight percent to about 4 weight percent potassium oleate. In some embodiments, the aqueous solution is a solution containing about 25 weight percent to about 40 weight percent potassium cocoate and about 2 weight percent to about 4 weight percent potassium oleate. This solution added to the cleanser in an amount of about 5 weight percent to about 40 weight percent provides from about 1.25 weight percent to about 18 weight percent potassium cocoate by total weight of the cleanser. For example, in some embodiments, the cleanser includes from 15 weight percent to 40 weight percent of the aqueous solution to provide from about 3.75 weight percent to about 18 weight percent potassium cocoate by total weight of the cleanser. In some embodiments, the cleanser includes from 25 weight percent to 40 weight percent of the aqueous solution to provide from about 6.25 weight percent to about 18 weight percent potassium cocoate by total weight of the cleanser. In some embodiments, the cleanser includes from 35 weight percent to 40 weight percent of the aqueous solution to provide from about 9 weight percent to about 18 weight percent potassium cocoate by total weight of the cleanser. In some embodiments, the cleanser includes about 37 weight percent of the aqueous solution to provide from about 9 weight percent to about 15 weight percent potassium cocoate by total weight of the cleanser. In some embodiments, the cleanser contains about 2 weight percent, about 4 weight percent, about 6 weight percent, about 8 weight percent, about 10 weight percent, about 12 weight percent, about 14 weight percent, about 16 weight percent, about 18 weight percent, or about 20 weight percent potassium cocoate by total weight of the cleanser.

As discussed above, some fatty acid salts are examples of a natural soap. Non-natural or non-naturally derived detergents are not natural soaps. Non-limiting examples of non-natural or non-naturally derived detergents include petroleum products and polyalkylene glycol-based, detergents and cleansers, e.g., polyethylene glycol-based and polypropylene glycol-based surfactants, polysorbates, and other non-naturally derived cationic surfactants, zwitterionic surfactants, anionic surfactants, and non-ionic surfactants. Naturally derived detergents and surfactants such as sulfates (e.g., sodium lauryl sulfate, sodium glucan sulfate), betaines, (e.g., cocamidopropyl betaine, lauryl betaine and coco-betaine), organic sulphonates (e.g., sodium C14-16 alpha olefin sulfonate), fatty acid and fatty alcohol glucosides (e.g., coco glucosides), alkanolamides (e.g., di and mono-alkanolamides such as coconut diethanol amide and coconut monoethanol amide) isethionates, taurates, and hydroxysultaines, are also not soaps.

In some embodiments, the cleanser may contain little to no non-natural or non-naturally derived detergents. For example, in some embodiments, the cleanser contains less than 5 weight percent of non-natural or non-naturally derived detergents by total weight of the cleanser. In some embodiments, the cleanser contains less than 1 weight percent of non-natural or non-naturally derived detergents by total weight of the cleanser. In some embodiments, the cleanser contains less than 0.1 weight percent of non-natural or non-naturally derived detergents by total weight of the cleanser. In some embodiments, the cleanser is free from non-natural or non-naturally derived detergents.

In some embodiments, the cleanser may contain little to no naturally derived detergents (other than the natural soap, i.e. a fatty acid salt). For example, in some embodiments, the cleanser contains less than 5 weight percent of naturally derived detergents (other than the natural soap) by total weight of the cleanser. In some embodiments, the cleanser contains less than 1 weight percent of naturally derived detergents (other than the natural soap) by total weight of the cleanser. In some embodiments, the cleanser contains less than 0.1 weight percent of naturally derived detergents (other than the natural soap) by total weight of the cleanser. In some embodiments, the cleanser is free from naturally derived detergents other than the natural soap.

In some embodiments, the cleanser may contain little to no sulfates. For example, in some embodiments, the cleanser contains less than 5 weight percent sulfates by total weight of the cleanser. In some embodiments, the cleanser contains less than 1 weight percent sulfates by total weight of the cleanser. In some embodiments, the cleanser contains less than 0.1 weight percent of sulfates by total weight of the cleanser. In some embodiments, the cleanser is free from sulfates.

### Moisturizers

The cleanser may contain a humectant based moisturizer or a natural/naturally derived skin conditioning agent to prevent skin drying.

The moisturizer can reduce skin irritation caused by the cleanser. Sugar-based moisturizers are a type of moisturizer effective for reducing skin irritation caused by a natural soap (e.g., a fatty acid salt). In some embodiments, the cleanser includes a sugar-based moisturizer. For example, in some embodiments, the moisturizer is a sugar-based moisturizer chosen from saccharide isomerate, trehalose, honey, sugar alcohols, glucose syrup, molasses and combinations thereof. In some embodiments, the composition includes alternative or additional moisturizers. The alternative or additional moisturizers can be natural or naturally derived humectants. In some embodiments, the cleanser contains from 0.1 weight percent to 5 weight percent of the moisturizer by total weight of the cleanser. For example, in some embodiments, the cleanser contains from 0.1 weight percent to 5 weight percent of a sugar-based moisturizer by total weight of the cleanser. In some embodiments, the cleanser contains from 0.1 weight percent to 5 weight percent of a sugar-based moisturizer chosen from saccharide isomerate, trehalose, honey, and combinations thereof by total weight of the cleanser. In some embodiments, the cleanser contains from 0.1 weight percent to 5 weight percent of saccharide isomerate by total weight of the cleanser.

In other embodiments, a superfatting or conditioning component can be included in the cleanser to function as a skin sealant to retain moisture. Such ingredients may enhance a barrier function to mitigate irritation and drying from the cleansing agents.

### Exfoliants

In some embodiments, the cleanser includes an exfoliant. Exfoliants can be useful for exfoliating skin when washed with the cleanser. In some embodiments, the cleanser includes an exfoliant chosen from volcanic sand, Argania spinosa shell powder, Bambusa arundinacea stem extract, and combinations thereof.

In some embodiments, the exfoliant may have particle sizes that range from 0 microns to 1,000 microns.

In some embodiments, the cleanser contains from 0.5 weight percent to about 5 weight percent of one or more exfoliants by total weight of the cleanser. In some embodiments, the cleanser contains from 2 weight percent to about 4 weight percent of one or more exfoliants by total weight of the cleanser. For example, in some embodiments, the cleanser contains about 1 weight percent of volcanic sand by total weight of the cleanser. In some embodiments, the cleanser contains about 1 weight percent of Argania spinosa shell powder by total weight of the cleanser. In some embodiments, the cleanser contains about 1.5 weight percent to about 2.5 weight percent of Bambusa arundinacea stem extract by total weight of the cleanser. In some embodiments, the cleanser contains about 3.25 weight percent of a combination of volcanic sand and Bambusa arundinacea stem extract by total weight of the cleanser. In some embodiments, the cleanser contains about 1 weight percent of volcanic sand and about 2.25 weight percent of Bambusa arundinacea stem extract by total weight of the cleanser. In some embodiments, the cleanser contains about 2.5 weight percent of a combination of Argania spinosa shell powder and Bambusa arundinacea stem extract by total weight of the cleanser. In some embodiments, the cleanser contains about 1 weight percent of Argania spinosa shell powder and about 1.5 weight percent of Bambusa arundinacea stem extract by total weight of the cleanser.

### Fragrances

In some embodiments, the cleanser includes a fragrance. Any suitable fragrance can be added; natural or naturally derived fragrances are preferred. The fragrance component can be a single fragrance compound, a mixture of fragrance compounds, a natural oil or extract, or a combination of natural oils and extracts. In some embodiments, the cleanser contains from 0.1 weight percent to about 3 weight percent of fragrance by total weight of the cleanser. In some embodiments, the cleanser contains from 1.5 weight percent to about 2 weight percent of fragrance by total weight of the cleanser.

In some embodiments, the cleanser includes components that assist in depositing, binding, or locking fragrances to the skin. These ingredients provide for a longer lasting presence on the skin. Ingredients that assist in these properties (also described elsewhere herein) include medium chain terminal diols, e.g., ethylhexylglycerin and similar compounds, and cationic skin conditioners, e.g., Hydroxypropyl Guar Hydroxypropyltrimonium Chloride.

### Chelating Agents

In some embodiments, the cleanser includes a chelating agent. Chelation can improve, for example, rheological properties, detergent quality, rinse properties, and/or skin irritation properties of the cleanser. Chelating agents may improve some properties of a cleanser containing a natural soap by, for example, chelating heavy metal ions that exist in tap water and are common causal factors of soap film and scum. Calcium and magnesium are common heavy metal ions in tap water. Chelation of calcium and magnesium can delay the counter ion exchange (e.g., from potassium to calcium) in the fatty acid salts that occurs during rinsing of the natural soap with tap water. Chelating agents also chelate iron and other ions that may negatively impact properties of the cleanser in use, for example when rinsing the soap from the skin.

In some embodiments, the chelating agent is a phytic acid derivative or other natural or naturally derived chelating agent, or synthetic agent. In some embodiments, the cleanser includes a chelating agent chosen from trisodium dicarboxymethyl alaninate, ethylenediaminetetraacetic acid (EDTA), trisodium ethylenediamine disuccinate (EDDS), tetrasodium glutamate diacetate, and combinations thereof. In some embodiments, the cleanser contains from 0.01 weight percent to 1 weight percent of the chelating agent by total weight of the cleanser. In some embodiments, the cleanser contains from 0.01 weight percent to 1 weight percent of a chelating agent chosen from trisodium dicarboxymethyl alaninate, ethylenediaminetetraacetic acid, trisodium ethylenediamine disuccinate, tetrasodium glutamate diacetate, and combinations thereof by total weight of the cleanser. In some embodiments, the cleanser contains from 0.01 weight percent to 1 weight percent of trisodium dicarboxymethyl alaninate by total weight of the cleanser. In some embodiments, the cleanser contains from 0.1 weight percent to 1 weight percent of the chelating agent by total weight of the cleanser. In some embodiments, the cleanser contains from 0.1 weight percent to 1 weight percent of a chelating agent chosen from trisodium dicarboxymethyl alaninate, ethylenediaminetetraacetic acid, trisodium ethylenediamine disuccinate, tetrasodium glutamate diacetate, and combinations thereof by total weight of the cleanser. In some embodiments, the cleanser contains from 0.1 weight percent to 1 weight percent of trisodium dicarboxymethyl alaninate by total weight of the cleanser. In some embodiments, the cleanser contains from 0.2 weight percent to 0.8 weight percent of the chelating agent by total weight of the cleanser. In some embodiments, the cleanser contains from 0.2 weight percent to 0.8 weight percent of a chelating agent chosen from trisodium dicarboxymethyl alaninate, ethylenediaminetetraacetic acid, trisodium ethylenediamine disuccinate, tetrasodium glutamate diacetate, and combinations thereof by total weight of the cleanser. In some embodiments, the cleanser contains from 0.2 weight percent to 0.8 weight percent of trisodium dicarboxymethyl alaninate by total weight of the cleanser.

### pH Adjusters

In some embodiments, the cleanser includes a pH adjuster, also referred to in the art as a pH modifier. The pH of a natural soap is typically basic (i.e., >7). A pH adjuster can be used to adjust the pH of the cleanser down up or down to a desired range or value. For example, a pH adjuster can be used to adjust the pH of the cleanser to from 6 to 11, 8 to 11, 6.5 to 11, 6.5 to 10, 8 to 10, from 8.5 to 10 or any other range. The pH adjuster can be an acid such as, e.g., citric acid. An amount of pH adjuster can be used to adjust the pH of the cleanser, if necessary, to a desired pH range. For example, in some embodiments, the cleanser contains from 0 to 1 weight percent pH adjuster by total weight of the cleanser. In some embodiments, the cleanser contains from 0.001 weight percent to 1 weight percent of the pH adjuster by total weight of the cleanser. In some embodiments, the cleanser contains from 0.001 weight percent to 1 weight percent of citric acid by total weight of the cleanser. In some embodiments, the cleanser contains from 0.001 weight percent to 0.5 weight percent of the pH adjuster by total weight of the cleanser. In some embodiments, the cleanser contains from 0.001 weight percent to 0.5 weight percent of citric acid by total weight of the cleanser.

### Skin Conditioning Agents

In some embodiments, the cleanser contains a skin conditioning agent. Skin conditioning agents can, for example, improve one or more skin properties when applied to the skin as a component of the cleanser. In some embodiments, the cleanser includes a skin conditioning agent chosen from a C6 to C20 diol, a quaternary ammonium modified guar gum, and combinations thereof. For example, in some embodiments, the cleanser includes ethylhexylglycerin and hydroxypropyl guar hydroxypropyltrimonium chloride as skin conditioning agents. Skin conditioning agents can also include emollients such as oils or ingredients commonly used as emollients to aid in super-fatting and skin conditioning.

The cleanser can include various amounts of the skin conditioning agents. For example, in some embodiments, the cleanser contains from 0.01 weight percent to 2 weight percent of skin conditioning agents by total weight of the cleanser. In some embodiments, the cleanser contains from 0.1 weight percent to 1 weight percent of ethylhexylglycerin by total weight of the cleanser. In some embodiments, the cleanser contains from 0.01 weight percent to 1 weight percent of hydroxypropyl guar hydroxypropyltrimonium chloride by total weight of the cleanser. In some embodiments, the cleanser contains from 0.1 weight percent to 1 weight percent of ethylhexylglycerin by total weight of the cleanser and from 0.01 weight percent to 1 weight percent of hydroxypropyl guar hydroxypropyltrimonium chloride by total weight of the cleanser.

### Humectants

In some embodiments, the cleanser contains a humectant. Humectants can reduce moisture loss of the cleanser and/or of the skin after the cleanser is applied to the skin. Exemplary humectants are glycols including, but not limited to, butylene glycol, propanediol, pentylene glycol, propylene glycol, and diglycerin. In some embodiments, the cleanser includes a humectant chosen from butylene glycol, propanediol, pentylene glycol, propylene glycol, diglycerin, glycerin, and combinations thereof. For example, in some embodiments, the cleanser includes glycerin as a humectant. The cleanser may include various amounts of the humectant. For example, in some embodiments, the cleanser contains from 0.1 weight percent to 15 weight percent of the humectant by total weight of the cleanser. In some embodiments, the cleanser contains from 0.1 weight percent to 15 weight percent of glycerin by total weight of the cleanser.

### Additional Optional Ingredients

In some embodiments, the cleanser includes one or more additional ingredients such as charcoal powder, hydrolyzed Gardenia florida extract, maltodextrin, or a combination thereof. Such components can improve, for example, the color, smell, consistency, and/or stability of the cleanser.

### Additional Cleanser Formulations and Properties

The cleanser can be formulated, for example, in accordance with any of the embodiments disclosed herein. In some embodiments, the cleanser contains at least 90 weight percent of naturally derived ingredients. In some embodiments, the cleanser contains at least 95 weight percent of naturally derived ingredients. In some embodiments, the cleanser contains at least 98 weight percent of naturally derived ingredients.

The cleanser can be formulated to reduce skin irritation as compared to a fatty acid salt administered to the skin alone. For example, in some embodiments, the cleanser passes the human repeat insult patch test for skin irritation.

In some embodiments, the cleanser includes from 5 weight percent to 40 weight percent of the fatty acid salt by total weight of the cleanser, from 0.1 weight percent to 5 weight percent of the moisturizer by total weight of the cleanser, from 0.01 weight percent to 1 weight percent of the chelating agent by total weight of the cleanser, from 0.001 weight percent to 1 weight percent of the pH adjuster by total weight of the cleanser, from 0.01 weight percent to 2 weight percent of skin conditioning agents by total weight of the cleanser, from 0.1 weight percent to 15 weight percent of the humectant by total weight of the cleanser, and from 0.01 weight percent to 2 weight percent of the viscosity modifier by total weight of the cleanser.

In some embodiments, the cleanser includes from 5 weight percent to 40 weight percent of potassium cocoate by total weight of the cleanser, from 0.1 weight percent to 5 weight percent of the saccharide isomerate by total weight of the cleanser, from 0.01 weight percent to 1 weight percent of trisodium dicarboxymethyl alaninate by total weight of the cleanser, from 0.001 weight percent to 1 weight percent of citric acid by total weight of the cleanser, from 0.1 weight percent to 1 weight percent of ethylhexylglycerin by total weight of the cleanser, from 0.01 weight percent to 1 weight percent of hydroxypropyl guar hydroxypropyltrimonium chloride by total weight of the cleanser, from 0.1 weight percent to 15 weight percent of glycerin by total weight of the cleanser, and from 0.01 weight percent to 2 weight percent of xanthan gum by total weight of the cleanser.

In some embodiments, the cleanser includes from 5 weight percent to 40 weight percent of potassium cocoate by total weight of the cleanser, from 0.1 weight percent to 5 weight percent of saccharide isomerate by total weight of the cleanser, from 0.01 weight percent to 1 weight percent of trisodium dicarboxymethyl alaninate by total weight of the cleanser, from 0.001 weight percent to 1 weight percent of citric acid by total weight of the cleanser, from 0.1 weight percent to 1 weight percent of ethylhexylglycerin by total weight of the cleanser, from 0.01 weight percent to 1 weight percent of hydroxypropyl guar hydroxypropyltrimonium chloride by total weight of the cleanser, from 0.1 weight percent to 15 weight percent of glycerin by total weight of the cleanser, and from 0.01 weight percent to 2 weight percent of xanthan gum by total weight of the cleanser; wherein the potassium cocoate, the saccharide isomerate, the trisodium dicarboxymethyl alaninate, the citric acid, the ethylhexylglycerin, the hydroxypropyl guar hydroxypropyltrimonium chloride, the glycerin, and the xanthan gum account for at least 90 weight percent of the cleanser by total weight of the cleanser.

In some embodiments, the cleanser includes from 5 weight percent to 40 weight percent of potassium cocoate by total weight of the cleanser, from 0.1 weight percent to 5 weight percent of saccharide isomerate by total weight of the cleanser, from 0.01 weight percent to 1 weight percent of trisodium dicarboxymethyl alaninate by total weight of the cleanser, from 0.001 weight percent to 1 weight percent of citric acid by total weight of the cleanser, from 0.1 weight percent to 1 weight percent of ethylhexylglycerin by total weight of the cleanser, from 0.01 weight percent to 1 weight percent of hydroxypropyl guar hydroxypropyltrimonium chloride by total weight of the cleanser, from 0.1 weight percent to 15 weight percent of glycerin by total weight of the cleanser, and from 0.01 weight percent to 2 weight percent of xanthan gum by total weight of the cleanser; wherein the potassium cocoate, the saccharide isomerate, the trisodium dicarboxymethyl alaninate, the citric acid, the ethylhexylglycerin, the hydroxypropyl guar hydroxypropyltrimonium chloride, the glycerin, and the xanthan gum account for at least 95 weight percent of the cleanser by total weight of the cleanser.

In some embodiments, the cleanser includes from 5 weight percent to 40 weight percent of potassium cocoate by total weight of the cleanser, from 0.1 weight percent to 5 weight percent of saccharide isomerate by total weight of the cleanser, from 0.01 weight percent to 1 weight percent of trisodium dicarboxymethyl alaninate by total weight of the cleanser, from 0.001 weight percent to 1 weight percent of citric acid by total weight of the cleanser, from 0.1 weight percent to 1 weight percent of ethylhexylglycerin by total weight of the cleanser, from 0.01 weight percent to 1 weight percent of hydroxypropyl guar hydroxypropyltrimonium chloride by total weight of the cleanser, from 0.1 weight percent to 15 weight percent of glycerin by total weight of the cleanser, and from 0.01 weight percent to 2 weight percent of xanthan gum by total weight of the cleanser; wherein the potassium cocoate, the saccharide isomerate, the trisodium dicarboxymethyl alaninate, the citric acid, the ethylhexylglycerin, the hydroxypropyl guar hydroxypropyltrimonium chloride, the glycerin, and the xanthan gum account for at least 99 weight percent of the cleanser by total weight of the cleanser.

In some embodiments, the cleanser is a body wash that includes ingredients as set forth in Table 1.

TABLE 1

| | | | Example weight % |
|---|---|---|---|
| | | Ingredient | range by total weight |
| Trade Name | INCI | Category | of the body wash |
| Neutrol MGDA | Trisodium | Chelating | Range 0.01%-1% |
| | Dicarboxymethyl | Agent | |
| | Alaninate | | |
| Citric Acid 50% | Citric Acid, Water | pH Adjuster | Range 0%-1% |
| (CITRIC ACID ANHY | | | |
| F6000 BAG114 - | | | |
| 16143596 & Water) | | | |
| ColaDet KC-40 | Potassium Cocoate | Natural Soap | Range 5%-40% |
| (Potassium Cocoate | | | |
| 25-40%, Potassium Oleate | | | |
| 2-4%, Water >50%) | | | |
| Sensiva SC 50 | Ethylhexylglycerin | Skin | Range 0.1%-1% |
| | | Conditioning | |
| Glycerin | Glycerin | Humectant | Range 0.1%-15% |
| Pentavitin | Saccharide | Moisturizer/ | Range 0.1%-5% |
| | Isomerate | Skin | |
| | | Conditioning | |
| Keltrol LAX-T | Xanthan Gum | Viscosity | Range 0.01%-2% |
| | | Modifier | |
| N-Hance HPCG | Hydroxypropyl Guar | Skin | Range 0.01%-1% |
| | Hydroxypropyl- | Conditioning | |
| | trimonium Chloride | | |
| Fragrance | | | 0-2% |
| Water | | | Balance |

In some embodiments, the cleanser is a body wash includes ingredients as set forth in Table 2.

TABLE 2

| | | | Example weight % |
|---|---|---|---|
| | | Ingredient | by total weight |
| Trade Name | INCI | Category | of the body wash |
| Neutrol MGDA | Trisodium | Chelating | 0.5% |
| | Dicarboxymethyl | Agent | |
| | Alaninate | | |
| Citric Acid 50% | Citric Acid, Water | pH Adjuster | 0.02% |
| (CITRIC ACID ANHY | | | |
| F6000 BAG114 - | | | |
| 16143596 & Water) | | | |
| ColaDet KC-40 | Potassium Cocoate | Natural Soap | 37% |
| (ingredient added to | | | |
| formula at 37% but | | | |
| breakdown is | | | |
| Potassium Cocoate | | | |
| 25-40%, Potassium Oleate | | | |
| 2-4%, Water >50%) | | | |
| Sensiva SC 50 | Ethylhexylglycerin | Skin | 0.5% |
| | | Conditioning | |
| Glycerin | Glycerin | Humectant | 8% |
| Pentavitin | Saccharide | Moisturizer/ | 0.5% |
| | Isomerate | Skin | |
| | | Conditioning | |
| Keltrol LAX-T | Xanthan Gum | Viscosity | 1.25% |
| | | Modifier | |
| N-Hance HPCG | Hydroxypropyl Guar | Skin | 0.4% |
| | Hydroxypropyl- | Conditioning | |
| | trimonium Chloride | | |
| Fragrance | | | 0-2% |
| Water | | | Balance |

In some embodiments, the cleanser is a hand soap includes ingredients as set forth in Table 3.

TABLE 3

| | | | Example weight % |
|---|---|---|---|
| | | Ingredient | by total weight |
| Trade Name | INCI | Category | of the hand soap |
| N-Hance HPCG | Hydroxypropyl Guar | Skin | 0.1% |
| | Hydroxypropyl- | Conditioning | |
| | trimonium Chloride | | |
| Citric Acid 50% FG | Citric Acid, Water | pH Adjuster | 0.05% |
| (RD-000337 & RD- | | | |
| 000004) | | | |
| Neutrol MGDA | Trisodium | Chelating | 0.2% |
| | Dicarboxymethyl | Agent | |
| | Alaninate, Water | | |
| ColaDet KC-40 | Potassium Cocoate, | Natural Soap | 27% |
| (ingredient added to | Potassium Oleate | | |
| formula at 37% but | | | |
| breakdown is | | | |
| Potassium Cocoate | | | |
| 25-40%, Potassium Oleate | | | |
| 2-4%, Water >50%) | | | |
| Pentavitin | Saccharide | Moisturizer/ | 0.5% |
| | Isomerate, Water, | Skin | |
| | Citric Acid, Sodium | Conditioning | |
| | Citrate | | |
| Glycerin | Glycerin | Humectant | 4.0% |
| Sensiva SC 50 | Ethylhexylglycerin | Skin | 0.5% |
| | | Conditioning | |
| Glycerin | Glycerin | Humectant | 4.0% |
| Keltrol LAX-T | Xanthan Gum | Viscosity | 1.0% |
| | | Modifier | |
| Fragrance | | | 0-2% |
| Water | | | Balance |

### Methods of Making a Cleanser

The cleanser can be prepared using various methods.

### Exemplary Method 1

In some embodiments, the cleanser is prepared by the following procedure:
preparing a first phase of the pH adjuster in water, optionally containing additives such as a skin conditioning agent,
preparing a second phase containing the fatty acid salt and the moisturizer and optionally including one or more of a humectant, a chelating agent or a skin conditioning agent,
adding the second phase to the first phase,
preparing a third phase containing the viscosity modifying agent and optionally a skin conditioning agent, and
adding the third phase to the combined first and second phase.

In embodiments that include an exfoliant, the exfoliant can be added as a fourth phase.

The pH can be checked and adjusted at different parts of the process and may be checked and adjusted as part of one or more steps.

### a Specific Example of Exemplary Method 1 is as Follows:

Phase 1. A skin conditioning agent (e.g., hydroxypropyl guar hydroxypropyltrimonium chloride) is added to water and mixed, followed by addition of a pH adjuster (e.g., citric acid) and mixing until there are no lumps. The mixture is heated to about 45° C.
Phase 2. The following ingredients are added sequentially with mixing at 45° C. with mixing to ensure incorporation of each ingredient before the next is added:
   Chelating agent, (e.g., trisodium dicarboxymethyl alaninate)
   Natural Soap (e.g., potassium cocoate)
   Humectant (e.g., Glycerin)
   Skin conditioning agent (e.g., ethylhexylglycerin)
   Moisturizer (e.g., saccharide isomerate)
   Fragrance
Phase 3. Premix Humectant (e.g., Glycerin) and a Viscosity Modifier (e.g., xanthan gum) and add to the solution from Phase 2 while mixing to avoid settling. Allow solution to cool while mixing and increase agitation as necessary until there are no lumps (about 40 minutes). Allow to cool to about 30° C.
Phase 4. (optional) While cooling to 30° C. add any exfoliant (e.g., volcanic sand, Argania spinosa shell powder, and/or Bambusa arundinacea stem extract) by sprinkling while mixing. Mix for 15-30 minutes and until well mixed, adjusting mixing speed as necessary to avoid aeration.

### Exemplary Method 2

### In Some Embodiments, the Cleanser is Prepared by the Following Procedure:

preparing an aqueous solution of a thickening agent,
adding an aqueous solution of a cleansing agent containing primarily a fatty acid salt to the aqueous solution of a thickening agent,
measuring the pH and adding a pH adjuster (if necessary) to obtain a pH ranging from to 6-11, and adding a moisturizer.

The pH can be adjusted at different steps of the process and may be adjusted as part of one or more steps.

### A Specific Example of Exemplary Method 2 is as Follows:

Step 1. Mix water and a skin conditioning agent (e.g., hydroxypropyl guar hydroxypropyltrimonium chloride).
Step 2. Add a pH adjuster (e.g., citric acid) to the mixture of step 1 and mix until the combination is uniform.
Step 3. (Optional) Mix a humectant (e.g., glycerin) and any optional ingredients (e.g., charcoal powder, hydrolyzed Gardenia florida extract, maltodextrin) to form a first premix and add the first premix to the mixture of step 2.
Step 4. Mix a humectant (e.g., glycerin) and a viscosity modifier (e.g., xanthan gum) to form a second premix and then slowly add the second premix to the mixture of step 3 (or step 2) to form a combined mixture.
Step 5. Heat the combined mixture of step 4 (e.g., at a temperature ranging from 70 degrees Celsius to 75 degrees Celsius).
Step 6. Add a fatty acid salt (e.g., potassium cocoate) and a chelating agent (e.g., trisodium dicarboxymethyl alaninate) to the combined mixture of step 5.
Step 7. Cool the mixture of step 6 (e.g., at a temperature ranging from 50 degrees Celsius to 60 degrees Celsius).
Step 8. Add a skin conditioning agent (e.g., ethylhexylglycerin) to the mixture of step 7.
Step 9. Adjust the pH (e.g., using the pH adjuster of step 2) to a desired pH range (e.g., 6-11 or 8.5-10). Optionally, cool the mixture while adjusting the pH (e.g., at a temperature ranging from 40 degrees Celsius to 45 degrees Celsius).
Step 10. Add a moisturizer (e.g., saccharide isomerate) to the mixture of step 9.
Step 11. Add a fragrance and any other additional ingredients not added in step 3 (e.g., a fragrance, charcoal powder, hydrolyzed Gardenia florida extract, maltodextrin, an exfoliant or a combination thereof) to the mixture of step 10.
Step 12. Optionally, filter the mixture of step 10 (e.g., through a 60-80 mesh filter).

Further aspects of the present disclosure are provided by the subject matter of the following clauses.

A cleanser comprising: a cleansing agent comprising a fatty acid salt; wherein
the cleanser is a gel having a dynamic viscosity at 25° C. and 1 atmosphere of pressure that ranges from 4,000 centipoise to 36,000 centipoise as determined with a Brookfield RV viscometer having a RV-5 spindle at 10 rpm,
the cleanser has a pH between 6 and 11, and
the cleanser comprises at least 90 weight percent of natural or naturally derived ingredients.

The cleanser of the preceding clause, comprising at least 95 weight percent of natural or naturally derived ingredients.

The cleanser of any preceding clause further comprising a moisturizer.

The cleanser of any preceding clause wherein the cleansing agent consists essentially of the fatty acid salt.

The cleanser of any preceding clause wherein the cleansing agent consists of the fatty acid salt.

A cleanser comprising: a fatty acid salt as the primary cleansing agent, and a moisturizer; wherein the cleanser is a gel with a pH between 6 and 11.

The cleanser of any preceding clause having a pH between 6.5 and 10.

The cleanser of the preceding clause having a pH between 8 and 11.

The cleanser of the preceding clause having a pH between 8.5 and 10.

The cleanser of the preceding clause having a pH of about 6.5.

The cleanser of any preceding clause having a dynamic viscosity at 25° C. and 1 atmosphere of pressure that ranges from 4,000 centipoise to 36,000 centipoise as determined with a Brookfield RV viscometer having a RV-5 spindle at 10 rpm.

The cleanser of any preceding clause having a measured viscosity that ranges from 8,000 to 32,000.

The cleanser of any preceding clause having a measured viscosity that ranges from 10,000 centipoise to 32,000 centipoise.

The cleanser of any preceding clause having a measured viscosity that ranges from 12,000 centipoise to 32,000 centipoise.

The cleanser of any preceding clause having a measured viscosity that ranges from 14,000 centipoise to 32,000 centipoise.

The cleanser of any preceding clause having a measured viscosity that ranges from 16,000 centipoise to 32,000 centipoise.

The cleanser of any preceding clause having a measured viscosity that ranges from 18,000 centipoise to 32,000 centipoise.

The cleanser of any preceding clause having a measured viscosity that ranges from 20,000 centipoise to 32,000 centipoise.

The cleanser of any preceding clause, wherein the cleanser comprises less than 5 weight percent of non-natural or non-naturally derived detergents by total weight of the cleanser.

The cleanser of any preceding clause, wherein the cleanser comprises less than 1 weight percent of non-natural or non-naturally derived detergents by total weight of the cleanser.

The cleanser of any preceding clause, wherein the cleanser comprises less than 0.1 weight percent of non-natural or non-naturally derived detergents by total weight of the cleanser.

The cleanser of any preceding clause, wherein the cleanser is free from non-natural or non-naturally derived detergents.

The cleanser of any preceding clause, wherein the cleanser comprises less than 1 weight percent sulfates by total weight of the cleanser.

The cleanser of any preceding clause, wherein the cleanser comprises less than 0.1 weight percent sulfates by total weight of the cleanser.

The cleanser of any preceding clause, wherein the cleanser is free from sulfates.

The cleanser of any preceding clause, wherein the cleanser is substantially free, i.e. contains less than 10 weight percent, less than 5 weight percent or less than 1 weight percent, of from non-natural or non-naturally derived detergents derived from petroleum products, polyalkylene glycol-based, e.g., polyethylene glycol-based and polypropylene glycol-based surfactants, detergents and cleansers, sulfates (e.g., sodium lauryl sulfate, sodium glucan sulfate), betaines, (e.g., cocamidopropyl betaine, lauryl betaine and coco-betaine), organic sulphonates (e.g., sodium C14-16 alpha olefin sulfonate), isethionates, taurates, hydroxysultaines, fatty acid and fatty alcohol glucosides (e.g., coco glucosides), alkanolamides (e.g., di and mono-alkanolamides such as coconut diethanol amide and coconut monoethanol amide), cationic surfactants, zwitterionic surfactants, anionic surfactants (other than fatty acid salts), non-ionic surfactants and polysorbates.

The cleanser of any preceding clause, wherein the cleanser is free from non-natural or non-naturally derived detergents derived from petroleum products, polyalkylene glycol-based, e.g., polyethylene glycol-based and polypropylene glycol-based surfactants, detergents and cleansers, sulfates (e.g., sodium lauryl sulfate, sodium glucan sulfate), betaines, (e.g., cocamidopropyl betaine, lauryl betaine and coco-betaine), organic sulphonates (e.g., sodium C14-16 alpha olefin sulfonate), isethionates, taurates, hydroxysultaines, fatty acid and fatty alcohol glucosides (e.g., coco glucosides), alkanolamides (e.g., di and mono-alkanolamides such as coconut diethanol amide and coconut monoethanol amide), cationic surfactants, zwitterionic surfactants, anionic surfactants (other than fatty acid salts), non-ionic surfactants and polysorbates.

The cleanser of any preceding clause, wherein the moisturizer is a sugar-based moisturizer.

The cleanser of any preceding clause, wherein the moisturizer is a sugar-based moisturizer chosen from saccharide isomerate, trehalose, honey, sugar alcohols, glucose syrup, molasses, and combinations thereof.

The cleanser of any preceding clause, wherein the fatty acid salt is an alkali metal salt.

The cleanser of any preceding clause, wherein the fatty acid salt is a potassium salt.

The cleanser of any preceding clause, wherein the fatty acid salt is chosen from potassium cocoate, potassium oleate, potassium palmate, and combinations thereof.

The cleanser of any preceding clause, wherein the fatty acid salt is potassium cocoate.

The cleanser of any preceding clause, wherein the fatty acid salt consists of one or more of a potassium fatty acid salts.

The cleanser of any preceding clause, wherein cleanser comprises from 1 weight percent to 40 weight percent of the fatty acid salt by total weight of the cleanser.

The cleanser of any preceding clause, wherein cleanser comprises from 1.25 weight percent to 20 weight percent of the fatty acid salt by total weight of the cleanser.

The cleanser of any preceding clause, wherein cleanser comprises from 3.75 weight percent to 20 weight percent of the fatty acid salt by total weight of the cleanser.

The cleanser of any preceding clause, wherein cleanser comprises from 6.25 weight percent to 20 weight percent of the fatty acid salt by total weight of the cleanser.

The cleanser of any preceding clause, wherein cleanser comprises from 9 weight percent to 15 weight percent of the fatty acid salt by total weight of the cleanser.

The cleanser of any preceding clause, wherein the fatty acid salt is added as an aqueous solution.

The cleanser of the preceding clause, wherein aqueous solution comprises from about 25 weight percent to about 50 weight percent of the fatty acid salt.

The cleanser of any preceding clause, further comprising a chelating agent.

The cleanser of any preceding clause, further comprising a chelating agent chosen from trisodium dicarboxymethyl alaninate, ethylenediaminetetraacetic acid, trisodium ethylenediamine disuccinate, tetrasodium glutamate diacetate, and combinations thereof.

The cleanser of any preceding clause, further comprising a pH adjuster.

The cleanser of any preceding clause, further comprising citric acid as a pH adjuster.

The cleanser of any preceding clause, further comprising a skin conditioning agent.

The cleanser of any preceding clause, further comprising a skin conditioning agent chosen from a C6 to C20 diol, a quaternary ammonium modified guar gum, and combinations thereof.

The cleanser of any preceding clause, further comprising ethylhexylglycerin and hydroxypropyl guar hydroxypropyltrimonium chloride as skin conditioning agents.

The cleanser of any preceding clause, wherein the skin conditioning agent is an emollient.

The cleanser of any preceding clause, further comprising an oil as a skin conditioning agent.

The cleanser of any preceding clause, further comprising a skin conditioning agent to aid in super-fatting and skin conditioning.

The cleanser of any preceding clause, further comprising a viscosity modifier chosen from water soluble polysaccharides, gums, complex starches, cellulosic thickeners, acrylate-based polymers, and combinations thereof.

The cleanser of any preceding clause, further comprising xanthan gum as a viscosity modifier.

The cleanser of any preceding clause, further comprising a viscosity modifier consisting essentially of xanthan gum.

The cleanser of any preceding clause, further comprising a viscosity modifier consisting of xanthan gum.

The cleanser of any preceding clause, further comprising a humectant.

The cleanser of any preceding clause, further comprising a humectant chosen from butylene glycol, propanediol, pentylene glycol, propylene glycol, diglycerin, glycerin, and combinations thereof.

The cleanser of any preceding clause, further comprising glycerin as a humectant.

The cleanser of any preceding clause, further comprising an exfoliant.

The cleanser of any preceding clause, further comprising an exfoliant chosen from volcanic sand, Argania spinosa shell powder, Bambusa arundinacea stem extract, and combinations thereof.

The cleanser of any preceding clause, further comprising a fragrance.

The cleanser of any preceding clause, further comprising charcoal powder, hydrolyzed Gardenia florida extract, maltodextrin, or a combination thereof.

The cleanser of any preceding clause, comprising from 5 weight percent to 40 weight percent of the fatty acid salt by total weight of the cleanser, from 0.1 weight percent to 5 weight percent of the moisturizer by total weight of the cleanser, from 0.01 weight percent to 1 weight percent of the chelating agent by total weight of the cleanser, from 0.001 weight percent to 1 weight percent of the pH adjuster by total weight of the cleanser, from 0.01 weight percent to 2 weight percent of skin conditioning agents by total weight of the cleanser, from 0.1 weight percent to 15 weight percent of the humectant by total weight of the cleanser, and from 0.01 weight percent to 2 weight percent of the viscosity modifier by total weight of the cleanser.

The cleanser of any preceding clause, comprising from 5 weight percent to 40 weight percent of potassium cocoate by total weight of the cleanser, from 0.1 weight percent to 5 weight percent of the saccharide isomerate by total weight of the cleanser, from 0.01 weight percent to 1 weight percent of trisodium dicarboxymethyl alaninate by total weight of the cleanser, from 0.001 weight percent to 1 weight percent of citric acid by total weight of the cleanser, from 0.1 weight percent to 1 weight percent of ethylhexylglycerin by total weight of the cleanser, from 0.01 weight percent to 1 weight percent of hydroxypropyl guar hydroxypropyltrimonium chloride by total weight of the cleanser, from 0.1 weight percent to 15 weight percent of glycerin by total weight of the cleanser, and from 0.01 weight percent to 2 weight percent of xanthan gum by total weight of the cleanser.

The cleanser of any preceding clause, comprising from 5 weight percent to 40 weight percent of potassium cocoate by total weight of the cleanser, from 0.1 weight percent to 5 weight percent of saccharide isomerate by total weight of the cleanser, from 0.01 weight percent to 1 weight percent of trisodium dicarboxymethyl alaninate by total weight of the cleanser, from 0.001 weight percent to 1 weight percent of citric acid by total weight of the cleanser, from 0.1 weight percent to 1 weight percent of ethylhexylglycerin by total weight of the cleanser, from 0.01 weight percent to 1 weight percent of hydroxypropyl guar hydroxypropyltrimonium chloride by total weight of the cleanser, from 0.1 weight percent to 15 weight percent of glycerin by total weight of the cleanser, and from 0.01 weight percent to 2 weight percent of xanthan gum by total weight of the cleanser; wherein the potassium cocoate, the saccharide isomerate, the trisodium dicarboxymethyl alaninate, the citric acid, the ethylhexylglycerin, the hydroxypropyl guar hydroxypropyltrimonium chloride, the glycerin, and the xanthan gum account for at least 90 weight percent of the cleanser by total weight of the cleanser.

The cleanser of any preceding clause, comprising from 5 weight percent to 40 weight percent of potassium cocoate by total weight of the cleanser, from 0.1 weight percent to 5 weight percent of saccharide isomerate by total weight of the cleanser, from 0.01 weight percent to 1 weight percent of trisodium dicarboxymethyl alaninate by total weight of the cleanser, from 0.001 weight percent to 1 weight percent of citric acid by total weight of the cleanser, from 0.1 weight percent to 1 weight percent of ethylhexylglycerin by total weight of the cleanser, from 0.01 weight percent to 1 weight percent of hydroxypropyl guar hydroxypropyltrimonium chloride by total weight of the cleanser, from 0.1 weight percent to 15 weight percent of glycerin by total weight of the cleanser, and from 0.01 weight percent to 2 weight percent of xanthan gum by total weight of the cleanser; wherein the potassium cocoate, the saccharide isomerate, the trisodium dicarboxymethyl alaninate, the citric acid, the ethylhexylglycerin, the hydroxypropyl guar hydroxypropyltrimonium chloride, the glycerin, and the xanthan gum account for at least 95 weight percent of the cleanser by total weight of the cleanser.

The cleanser of any preceding clause, comprising from 5 weight percent to 40 weight percent of potassium cocoate by total weight of the cleanser, from 0.1 weight percent to 5 weight percent of saccharide isomerate by total weight of the cleanser, from 0.01 weight percent to 1 weight percent of trisodium dicarboxymethyl alaninate by total weight of the cleanser, from 0.001 weight percent to 1 weight percent of citric acid by total weight of the cleanser, from 0.1 weight percent to 1 weight percent of ethylhexylglycerin by total weight of the cleanser, from 0.01 weight percent to 1 weight percent of hydroxypropyl guar hydroxypropyltrimonium chloride by total weight of the cleanser, from 0.1 weight percent to 15 weight percent of glycerin by total weight of the cleanser, and from 0.01 weight percent to 2 weight percent of xanthan gum by total weight of the cleanser; wherein the potassium cocoate, the saccharide isomerate, the trisodium dicarboxymethyl alaninate, the citric acid, the ethylhexylglycerin, the hydroxypropyl guar hydroxypropyltrimonium chloride, the glycerin, and the xanthan gum account for at least 99 weight percent of the cleanser by total weight of the cleanser.

The cleanser of any preceding clause, wherein the cleanser passes the human repeat insult patch test for skin irritation. The cleanser of any preceding clause, wherein the cleanser comprises at least 98 weight percent of natural or naturally derived ingredients.

A method of making the cleanser of any preceding clause comprising the steps of
preparing an aqueous solution adding a viscosity modifier,
adding an aqueous solution of a cleansing agent comprising a fatty acid salt, and
obtaining a pH ranging from to 6-11 by optionally adding a pH adjuster, if necessary,
wherein the cleanser comprises at least 90 weight percent of natural or naturally derived ingredients and/or the fatty acid salt is the primary cleansing agent.

The method of the preceding clause, further comprising adding a moisturizer.

The method of the preceding clause, wherein the moisturizer is added to the aqueous solution of the cleansing agent.

The method of the preceding clause, wherein the viscosity modifier is added after the cleansing agent.

The method of the preceding clause, wherein the viscosity modifier is added to the aqueous solution of the cleansing agent and the moisturizer.

The method of any preceding clause, wherein the aqueous solution of the cleansing agent is added after the viscosity modifier.

The method of the preceding clause, further comprising adding a moisturizer to the combined aqueous solution of the cleansing agent and the viscosity modifier.

The method of any preceding clause, wherein the aqueous solution of cleansing agent comprises 25-40 wt. % of the fatty acid salt.

The method of any preceding clause, wherein the fatty acid salt is a potassium fatty acid salt.

The method of any preceding clause, wherein the aqueous solution of cleansing agent comprises less than 5 weight percent of non-natural or non-naturally derived detergents in the cleanser gel.

The method of any preceding clause, wherein the viscosity modifier is added in an amount sufficient to provide the cleanser gel with a dynamic viscosity at 25° C. and 1 atmosphere of pressure that ranges from 4,000 centipoise to 36,000 centipoise as determined with a Brookfield RV viscometer having a RV-5 spindle at 10 rpm.

The method of any preceding clause, wherein the viscosity modifier comprises a polysaccharide gum.

The method of any preceding clause, wherein the viscosity modifier consists essentially of a polysaccharide gum.

The method of any preceding clause, wherein the thickening agent is xanthan gum.

The method of any preceding clause wherein the step of adding a viscosity modifier comprises preparing an aqueous solution of the viscosity modifier and one or more of a skin conditioning agent and a humectant.

The method of any preceding clause, wherein the aqueous solution of cleansing agent further comprises a chelating agent.

The method of any preceding clause, further comprising adding a fragrance.

The method of any preceding clause, wherein the step of obtaining a pH comprises obtaining a pH ranging from 8.5-10.

The method of any preceding clause, further comprising at least one of:
heating the aqueous solution of thickening agent to 70° C.-75° C. before adding the aqueous solution of cleansing agent;
maintaining a temperature of 70° C.-75° C. during addition of the aqueous solution of cleansing agent to the aqueous solution of thickening agent;
cooling to a temperature of 50° C.-60° C. after addition aqueous solution of cleansing agent and before the step of obtaining a pH; and
cooling to a temperature of 40° C.-45° C. before adding the moisturizer.

The method of any preceding clause, further comprising the step of adding an exfoliant.

Cleansers according to embodiments described herein provide numerous benefits over prior art cleansers. For example, the cleansers can be provided as stable gels that do not include synthetic surfactants. Thus, the cleansers can be made into products that are desirable for many users. Embodiments of the cleansers also provide other benefits, such as reduced skin irritation and easy foamability, which helps create easily dispensable products. Still further benefits are derived from the gelled nature of the product. This feature increases ease of dispensing and use as compared to traditional liquid soaps, which are thin and have no added ingredients for a smooth after feel or long lasting hydration. Those skilled in the art will recognize numerous other benefits of the embodiments beyond those expressly described herein.

The embodiments illustrated and discussed in this specification are intended only to teach those skilled in the art how to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of any and all claims supported herein and their equivalents, the invention may be practiced otherwise than as specifically described.

What is claimed is:

1. A body wash comprising: a cleansing agent that includes a fatty acid salt; wherein:
the body wash is a gel having a dynamic viscosity at 25° C. and 1 atmosphere of pressure that ranges from 4,000 centipoise to 36,000 centipoise as determined with a Brookfield RV viscometer having a RV-5 spindle at 10 rpm,
the body wash has a pH between 6 and 11, and
the body wash comprises at least 90 weight percent of natural or naturally derived ingredients.

2. The body wash of claim 1, wherein the fatty acid salt is the primary cleansing agent.

3. The body wash of claim 1, comprising at least 95 weight percent of natural or naturally derived ingredients.

4. The body wash of claim 1, wherein the fatty acid salt is a potassium salt.

5. The body wash of claim 1, wherein the body wash has a pH ranging from 6.5 to 10.

6. The body wash of claim 1, further comprising a viscosity modifier chosen from water soluble polysaccharides, gums, complex starches, cellulosic thickeners, acrylate-based polymers, and combinations thereof.

7. The body wash of claim 1, wherein the viscosity modifier comprises xanthan gum.

8. The body wash of claim 1, wherein the body wash comprises less than 1 weight percent sulfates by total weight of the body wash.

9. The body wash of claim 1, wherein the body wash is free from sulfates.

10. The body wash of claim 1, further comprising a moisturizer.

11. The body wash of claim 10, wherein the moisturizer is a sugar-based moisturizer chosen from saccharide isomerate, trehalose, honey, sugar alcohols, glucose syrup, molasses, and combinations thereof.

12. The body wash of claim 1, wherein the cleansing agent consists essentially of the fatty acid salt.

13. The body wash of claim 1, wherein the cleansing agent consists of the fatty acid salt.

14. A hand soap comprising: a cleansing agent that includes a fatty acid salt; wherein:
the hand soap is a gel having a dynamic viscosity at 25° C. and 1 atmosphere of pressure that ranges from 1,000 centipoise to 10,000 centipoise as determined with a Brookfield RV viscometer having a RV-4 spindle at 10 rpm,
the hand soap has a pH between 6 and 11, and
the hand soap comprises at least 90 weight percent of natural or naturally derived ingredients.

15. The hand soap of claim 14, wherein the fatty acid salt is the primary cleansing agent.

16. The hand soap of claim 14, comprising at least 95 weight percent of natural or naturally derived ingredients.

17. The hand soap of claim 14, wherein the fatty acid salt is a potassium salt.

18. The hand soap of claim 14, wherein the hand soap has a pH ranging from 6.5 to 10.

19. The hand soap of claim 14, further comprising a viscosity modifier chosen from water soluble polysaccharides, gums, complex starches, cellulosic thickeners, acrylate-based polymers, and combinations thereof.

20. The hand soap of claim 14, wherein the viscosity modifier comprises xanthan gum.

21. The hand soap of claim 14, wherein the hand soap comprises less than 1 weight percent sulfates by total weight of the hand soap.

22. The hand soap of claim 14, wherein the hand soap is free from sulfates.

23. The hand soap of claim 14, further comprising a moisturizer.

24. The hand soap of claim 23, wherein the moisturizer is a sugar-based moisturizer chosen from saccharide isomerate, trehalose, honey, sugar alcohols, glucose syrup, molasses, and combinations thereof.

25. The hand soap of claim 14, wherein the cleansing agent consists essentially of the fatty acid salt.

26. The hand soap of claim 14, wherein the cleansing agent consists of the fatty acid salt.
